(19) United States
(12) Patent Application Publication
(10) Pub. No.: US 2014/0128431 A1
(43) Pub. Date: May 8, 2014

(54) PHARMACEUTICAL COMPOSITION WITH IMPROVED BIOAVAILABILITY, SAFETY AND TOLERABILITY

(71) Applicant: Hoffmann-LaRoche Inc., Nutley, NJ (US)

(72) Inventors: Shalini M. Anand, Nutley, NJ (US); Ashish Chatterji, East Brunswick, NJ (US); Michael Allen Matchett, Montville, NJ (US); Harpreet K. Sandhu, West Orange, NJ (US); Navnit H. Shah, Clifton, NJ (US)

(73) Assignee: Hoffmann-LaRoche Inc., Nutley, NJ (US)

(21) Appl. No.: 13/854,248

(22) Filed: Apr. 1, 2013

Related U.S. Application Data
(60) Provisional application No. 61/619,635, filed on Apr. 3, 2012.

(51) Int. Cl. A61K 47/34; A61K 31/4709

(52) U.S. Cl. CPC A61K 47/34; A61K 31/4709
USPC 514/314

(57) ABSTRACT

The invention relates to solid dispersions of poorly soluble compounds formed by coprecipitation, resulting in improved bioavailability, safety and tolerability. The invention also relates to Purified Eudragit L100-55 used to prepare such solid dispersions.

### FIELD OF THE INVENTION

The present invention relates to the solid amorphous dispersion of N-{4-[6-tert-Butyl-5-methoxy-8-(6-methoxy-2-oxo-1,2-dihydro-pyridin-3-yl)-quinolin-3-yl]-phenyl}methane-sulfonamide which is a practically insoluble compound to improve its bio-availability, safety and tolerability.

### BACKGROUND OF INVENTION

The present invention relates to pharmaceutical composition comprising of the stabilized solid amorphous dispersion of extremely low-solubility compound which resulted in significantly enhanced dissolution and bioavailability over the crystalline form of the compound. Another aspect of the invention relates to stabilized amorphous formulation of the compound, obtainable by said process, while using the modified polymer in order to improve the safety and tolerability of the formulation at the target dose.

Many pharmaceutical compounds, particular that are highly potent possess poor solubility in water. Such drugs may be classified according to USP-NF as being sparingly soluble, slightly soluble, very slightly soluble, or insoluble in water. Many of these compounds are also poorly soluble in oils.

As used herein, the term “poorly soluble” when referring to a chemical compound in relation to its solubility in water or an oil, as defined in U.S. Pharmacopeia and National Formulary (USP-NF). According to this definition, solubility is stated in terms of the parts of the solvent needed to dissolve one part of the solute. A compound that is sparingly soluble in a particular solvent, such as water, requires 30-100 parts of the solvent to dissolve one part of the compound. A compound that is slightly soluble, requires 100-1000 parts of the solvent. A compound that is very slightly soluble requires 1000-10,000 parts of the solvent. A compound that is insoluble requires more than 10,000 parts of the solvent to dissolve one part of the solute.

The lack of solubility of such drugs, and the inability to obtain sufficiently high concentrations of drugs in solution in pharmaceutically acceptable carriers, is a serious problem for formulating these drugs and thereof limit the therapeutic benefit that can be achieved for such compounds. Lack of solubility is additionally a concern in the formulation of compounds for various different targets which need significantly high doses and need to establish very high safety margin over the therapeutic effective dose. Accordingly, a significant need existed for a method to increase the solubility of these drugs.

To improve the desired properties of poorly soluble drugs, many technologies have been developed, including but not limited to the following:

1. Salt Formation: This is the most widely used approach to increase solubility of weakly acidic or basic NCE's. (see e.g. Wadke, D. A. et al, Pharmaceutical Dosage Forms: Tablets, Vol. 1, 1989, pp 1-73). The solubility of salt is typically driven by the counter ion and selection of counter-ion is based on many parameters such as solubility, hygroscopicity and stability of the physical form. In spite of the numerous advantages associated with salt forms, developing a stable salt is not always feasible. In many cases, increased dissolution rate is difficult to achieve because of the reconversion of salts into their respective acid or base forms.

2. Particle size reduction: Due to their poor solubility, the absorption/bioavailability of some compounds is dissolution rate limited. A reduction in particle size improves the dissolution rate significantly, which provides better absorption potential and potentially leads to improved therapeutics. Wet milling (see e.g. U.S. Pat. No. 5,494,683) and Nano-technology (see e.g. PCT Int. Appl. WO 2004022100) are two examples of the techniques that can be applied to poorly water soluble drugs. Although these conventional methods have been used commonly to increase dissolution rate of drug, there are practical limitations as the desired bioavailability enhancement may not always be achieved simply by particle size reduction. Also, agglomeration due to increased surface energy or poor wetting can overcome any benefit of reduced particle size.

3. Lipid formulation: Poorly soluble drugs may dissolve in lipid based vehicle at much higher concentration than in aqueous media. After being dosed, the lipid formulation is dispersed in gastric and intestinal fluid, which provides a large surface area for the drug to diffuse from its solution in lipid to the gastric or intestinal fluid. The high solubility of the drug in the lipid formulation provides the strong driving force for the diffusion. Self-emulsifying drug delivery system (SEDDS) is one example. Depending on the selection of the lipid vehicle, the resulting aqueous dispersion may yield very fine or crude emulsion (see e.g. U.S. Pat. Nos. 5,969,160; 6,057,289; 6,555,558 and 6,638,522). Some constrains for these formulation techniques comes from insufficient drug solubility in lipid vehicles, physical in-stability (e.g. polymorph crystallization with reduced solubility) etc.

4. Cosolvents: Cosolvents can be used in the formulations of poorly water soluble drugs for better solubilization and consequently better bioavailability (see e.g. U.S. Pat. No. 6,730,679).

5. Complexation: Complexation using carriers such as cyclodextrins is another approach that can be used for solubilization and screening of poorly soluble compounds (see e.g. U.S. Patent No. 2006128653). Despite the significant promise of cyclodextrins, these solubilizing aids have their own limitations, the use of cyclodextrins in a formulation can limit the dose level, depending on the toxicity and solubilization potential of the carrier (see e.g. Uchenna Agu, R. et al Int. J. Pharm. 193, 219-226, 2000; Rao, V. M., Stella, V. J. J. Pharm. Sci. 92, 927-932, 2003).

6. Solid dispersion: In recent years, solid dispersions have attracted attention in the field of oral preparations, especially for the poorly soluble compounds. Solid dispersion technologies involve stabilization of the drug in its amorphous form, within a carrier matrix. The amorphous form allows faster dissolution of the drug and is particularly promising for orally administered drugs (because of the wider choices of carrier matrices). However, to use this technology effectively identification of an appropriate carrier that is compatible with the drug is necessary. Several techniques have been developed to prepare solid dispersions, including co-precipitation (see e.g. U.S. Pat. Nos. 5,985,326 and 6,350,786), fusion, spray-drying (see e.g. U.S. Pat. No. 7,008,640), and hot-melt extrusion (see e.g. U.S. Pat. No. 7,081,255). All these techniques provide a highly dispersed drug molecule in polymer matrix, which improve the dissolution of the drug from the dispersion. The solid dispersions prepared from different methods may differ in properties, such as porosity, surface area, density, stability, hygroscopicity, dissolution and therefore bioavailability. However, there is no evidence in the literature suggesting the superiority of one method over another to achieve the desired pharmacokinetic profile, particularly better dose proportionality.

While some of these techniques are well known, most of them provide a number of unique challenges and can't be applicable to the brick dust like compounds i.e. with very high melting point and practically no solubility in any of the organic solvents.

Furthermore, the amorphous solid dispersions are high energy formulations which present additional challenges since they are, by nature, thermodynamically unstable. Consequently, their successful development depends in good measure on the understanding of the specific interactions responsible for their stabilization (Serajuddin, A. T. M. J. Pharm. Sci. 1999, 88, 1058-1066, Janssens, S.; Van den Mooter, G. J. Pharm. Phamacol. 2009, 61, 1571-1586.)

### BRIEF DESCRIPTION OF THE FIGURES

FIG. 1: Summary of Compound A bioavailability in rats at various different doses of PEG-Labrasol formulation (Minitox study #)

FIG. 2: XRD Pattern for crystalline API (Form I, II, III)

FIGS. 3A and B: XRD pattern of the Formulation 17 (MBP with HPMC-AS) and Formulation 19 (MBP with Eudragit L100-55) as is vs. under stress conditions

FIG. 4: Dissolution profile of crystalline vs. various amorphous formulations

FIGS. 5A and B: Relative bioavailability profile A) Comparative Bioavailability with crystalline formulation vs. amorphous formulation with Eudragit L100-55 vs. MBP with HPMC-AS
B) Dose proportional increase in exposure with Eudragit L100-55 based MBP formulation

FIG. 6: FT-IR Specta of crystalline API vs. HPMC-AS MBP and MBP with Eudragit L100-55

FIG. 7: FT-IR spectra of HPC-AS MBP and Eudragit L100-55 MBP under stress conditions

FIG. 8: Dissolution of Eudragit L100-55 MBP under acidic conditions

FIGS. 9A, B and C: In-vivo performance of MBP with Purified Eudragit L100-55

### DESCRIPTION OF RELATED ART

The preparation of the crystalline form of N-{4-[6-tert-Butyl-5-methoxy-8-(6-methoxy-2-oxo-1, 2-dihydro-pyridin-3-yl)-quinolin-3-yl]-phenyl}methane-sulfonamide API has been described in U.S. Patent Publication No. 2010/0311760 A1. The patent describes various methods for making the crystalline forms of the API and the mechanism of action for the molecule.

U.S. Pat. No. 6,350,786 discloses pharmaceutical compositions comprising of amorphous dispersion of various different compounds i.e. Compound I, Tolcapone, Accutane, Saquinavir and several others, obtained by using microprecipetation bulk Powder (MBP) technology. The MBP technology was found to be widely applicable and several different polymers i.e. Eudragit® L100-55, Eudragit® L100, Hydroxypropylmethylcellulose phthalate (HP-50) or Eudragit® S100 were found to be successful in generating stable amorphous dispersion for these drugs.

US Patent Application No. US2010/029489 describes pharmaceutical composition of PLX4032 with HPMC-AS using MBP technology. US Patent Application No. US2009/145999 entails amorphous composition of PLX4032 with co-povidone polymer via hot melt extrusion process, indicating that amorphous dispersion of RO5185426 can be feasible with different methods and polymers.

U.S. patent application Ser. No. 12/902,186 specifies a pharmaceutical composition of low melting drug HEP with HPMC-AS using MBP and HME technology, where amorphous dispersion via HME process showed slightly improved pharmaco-kinetic behavior over MBP formulation.

In U.S. Pat. No. 6,350,786, solid dispersions using water-insoluble ionic polymers with a molecular weight greater than 80,000 D are disclosed to provide a stable amorphous formulation.

U.S. Pat. No. 6,548,555 describes the use of ionic polymers, including hydroxypropyl-methyl cellulose acetate succinate (HPMCAS), to prepare solid dispersions for improved solubility and better bioavailability.

Kondo et al showed improved oral absorption of poorly soluble drugs in enteric co-precipiates (see e.g. J Pharm Sciences, 83(4) 1994). The polymer used in this preparation was hydroxyl propyl methyl cellulose phthalate and the co-precipitates were prepared by solvet evaporation method followed by drying at 80° C. Based on dissolution data, such co-precipitates systems solubilized by co solvents or solid dispersion approaches, may revert back to crystalline form, resulting in loss of bioavailability at higher dose.

While Microprecipitation has been utilized for the stabilization of many drug substances in the solid state, it may not be possible to satisfactorily tailor the pharmacokinetic profile of such poorly soluble compounds, particularly the dose dependent exposure, which is very important to manage the safety and efficacy of the compound. These supersaturated formulations may revert back to crystalline form upon storage or under stress conditions, resulting in loss of bioavailability. The polymer and process selection for amorphous dispersion found to play critical role in stabilizing those dispersion. Thus, there is no absolute method to a priori judge whether a given polymer or process will provide adequate stability of the amorphous dispersion.

### SUMMARY OF THE INVENTION

N-{4-[6-tert-Butyl-5-methoxy-8-(6-methoxy-2-oxo-1,2-dihydro-pyridin-3-yl)-quinolin-3-yl]-phenyl}methane-sulfonamide (Compound A) is a potent compound for the treatment of Hepatitis-C infection. But it possess several associated challenges such as, high melting point (>270° C.), unfavorable physico-chemical properties (practically insoluble in aqueous and most of the organic solvents) and high dose projections (>1 g/day). Due to these challenges, the development of suitable dosage formulation using conventional methods was not feasible and resulted in sub-optimal bioavailability. The unique feature of this invention is that it has been unexpectedly found that out of several screened carriers for amorphous dispersion formulations; only one polymer was able to demonstrate sufficient stability for amorphous formulation and significant increase in the bioavailability, enabling the use of amorphous formulation for future studies. The key features of the invention are:
a) Preparation of solid dispersion of N-{4-[6-tert-Butyl-5-methoxy-8-(6-methoxy-2-oxo-1,2-dihydro-pyridin-3-yl)-quinolin-3-yl]-phenyl}methane-sulfonamide (Compound A) using MBP technology,
b) Use of Eudragit L100-55 polymer at the level of 60% to 99%,
c) Drug loading of 1-40% in the final product,

In another embodiment, the amorphous dispersion can be comprised of Compound A API and purified Eudragit L100-55, to improve the safety and tolerability, associated with the polymer at target dose in the pre-clinical and clinical studies.

The purified Eudragit L100-55 contains lower amount of surfactant, specially sodium lauryl sulphate (SLS), I.E. <0.1%, and the removal of surfactant didn't pose any effect on the in-vitro and in-vivo performance of amorphous formulation.

### DETAILED DESCRIPTION OF THE INVENTION

The present invention relates to stabilized solid dispersion of N-{4-[6-tert-Butyl-5-methoxy-8-(6-methoxy-2-oxo-1,2-dihydro-pyridin-3-yl)-quinolin-3-yl]-phenyl}methane-sulfonamide (Compound A) prepared by Micro-precipitated Bulk Powder (MBP) process which have an enhanced dissolution rate and an significantly improved bioavailability.

The stable compositions comprises of about 1% to 40%, preferably from 1% to 30% of the API molecularly dispersed in 60% to 99% of methyl methacrylate based copolymer i.e., Eudragit L100-55 which is a copolymer of methacrylate and ethyl acrylate. Most preferably the stabilized amorphous dispersion composition of HCV-4 of the present invention comprises no significant amounts of crystalline API, as demonstrated by amorphous X-ray diffraction of above compositions.

The active ingredient having the chemical name of N-{4-[6-tert-Butyl-5-methoxy-8-(6-methoxy-2-oxo-1,2-dihydro-pyridin-3-yl)-quinolin-3-yl]-phenyl}-methanesulfonamide, (Compound A), can be represented by the following structural and nomenclature formula:

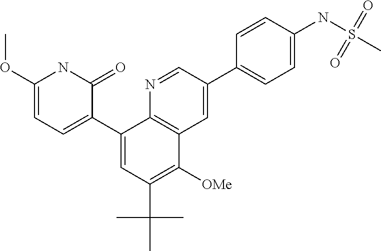 [SMILES: COc1ccc(-c2cc(C(C)(C)C)c(OC)c3cc(-c4ccc(NS(C)(=O)=O)cc4)cnc23)c(=O)[nH]1]

N-{4-[6-tert-Butyl-5-methoxy-8-(6-methoxy-2-oxo-1,2-dihydro-pyridin-3-yl)-quinolin-3-yl]-phenyl}-methanesulfonamide.

Compound A belongs to the class of non-nucleoside analog for the treatment of Hepatitis-C infection via inhibiting RNA-dependent RNA polymerase The RNA polymerase is required for viral replication and thus represents an attractive target for the treatment of Hepatitis-C. Direct acting antiviral (DAA) combinations are the next evolution in HCV treatment and Polymerase non-nucleoside inhibitors (NNI) is expected to be successful components of DAA combinations.

Currently combination therapy with Ribavarin and interferon-a is the optimal therapy for HCV. But unfortunately, the combination therapy produces several side effect i.e rashes, hemolytic anemia which poses significant clinical challenges. Compound A has been targeted to be the part of interferon/ribavarin-free combination therapy for the treatment of Hepatitis-C.

The crystalline form of Compound A API has a melting point of approximately 273° C. and possess very low aqueous solubility (<1 μg/ml) at physiological pHs (from pH 1.5-8.0), consequently very low bioavailability. The compound is moderately permeable as determined with the Caco-2 assay value of 4.5×10-6 cm/s. Targeted high doses/frequency of dosing for this series of compounds, led to the categorization of Compound A as BCS class IV compound (low solubility/low permeability).

Extremely low solubility/bioavailability pose challenges to attaining desirable exposure and safety margins for Compound A. Since the low bioavailability of hydrophobic drugs with extremely low water solubility can be a serious problem, different approaches have been taken to achieve the desirable high levels of drug solubility and dissolution rate.

### Crystalline Formulation Approaches

Below are the details (examples 1-4) of various different formulation approaches with crystalline form or salt form of the compound. Table 1 illustrates the relative bio-availability obtained with those formulation approaches.

The crystalline formulations were produced as follows:

### EXAMPLE 1

### Crystalline Wet Milled Suspension

Crystalline suspension was prepared by dispersing the drug in aqueous based klucel vehicle comprising of 2% hydroxypropyl cellulose, 0.1% polysorbate 80, 0.09% methylparaben, 0.01% propylparaben, and purified water. Acetate buffer is added to adjust to pH 3.5±0.5. The suspension was sonicated for 30 mins. The final achived median particle size was 100 μm (d_{0.5}).

### EXAMPLE 2

### Nanosuspension

The Nano suspension was prepared using nano mill 01 system, by elan (Model no: NM-026). The HBr salt of the drug was dispersed in aqueous based klucel vehicle comprising and passed through the nano-mill. The final achieved median particle size was ˜300 nm (d_{0.5}).

### EXAMPLE 3

### PEG-Labrasol Formulation

The HBr salt of the API was dispersed in TG31 vehicle containing 50:50 (w/w) of PEG-400/Labrasol and was sonicated for about 20 minutes. The resulting solution was stored at −20° C. and was warmed to room temperature prior to dosing.

### EXAMPLE 4

### Cyclodextrin Based Formulation

The captisol formulation of Compound A was formulated using the HBr salt of the API. The HBr salt was dispersed in 30% (w/w) sulfobutylether-beta-cyclodextrin solution and sonicated for 10-15 mins. Yellow-colored clear solution was obtained up to 60 mg/ml concentration.

The above formulation was administered in doses as high up to 1000 mg/kg, to achieve the 20-30× safety margin window required to establish the safe dose for this series of compounds. The limited solubility of the HBr salt in captisol led to suspension formulation at higher doses beyond 300 mg/kg.

The crystalline form of the compound didn't show any solubility improvement over HBr salt of the compound.

TABLE 1
Comparison of Rat PK data for various crystalline formulations
at the dose level of 30 mg/kg

| | | | AUC/AUC_{eff} |
|---|---|---|---|
| | | AUC/Dose | (Safety Margin |
| Formulation | API Form | (μg · h/mL) | Window) |
| Wet milled Suspension | HBr Salt | 1.7 | <2 |
| Nano Suspension | HBr Salt | 0.8 | <2 |
| PEG-Labrasol formulation | HBr Salt | 18.6 | 2.6 |
| Cyclodextrin based | HBr Salt | 21 | 2.8 |
| formulation | | | |

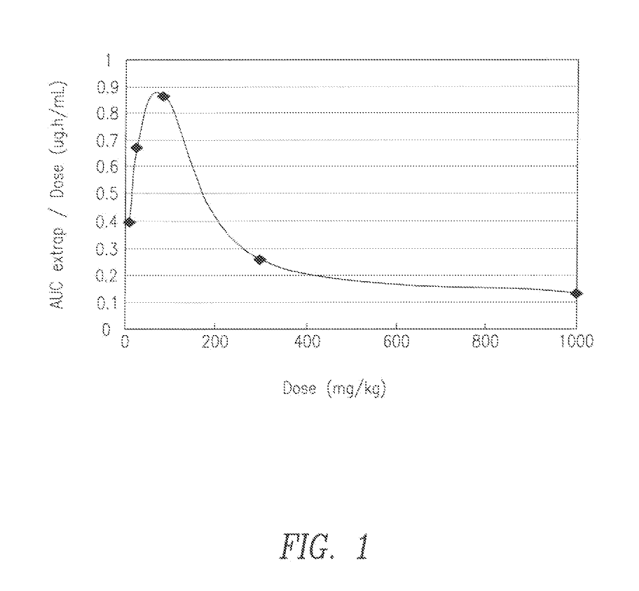

The desired efficacious exposure of the compound was 4.0-7.0 μg·h/ml. The wet milled suspension resulted in dose-normalized exposure of about 1.6 μg·h/mL whereas nano-suspension provided even further lower exposure i.e. 0.8 μg·h/ml. PEG/Labrasol formulation led to the bioavailability of 18.6 μg·h/mL, with no further improvement at higher doses. The captisol formulation led to variability and saturation in PK at higher doses as shown in FIG. 1.

### Amorphous Formulation Approaches

It was found that amorphous solid dispersion of Compound A exhibited significantly higher bioavailability than crystalline or salt form of the compound.

Various available technologies were evaluated to generate the suitable amorphous formulation i.e. spray drying, hot melt extrusion, and microprecipetated bulk powder technology, as shown in examples 5-20.

The various carriers which were screened includes, hydroxypropyl methylcellulose, hydroxypropyl methyl cellulose-acetyl succinate, Hydroxypropymethyl cellulose phthalate, cellulose acetate phthalate; Kollidon PVP, Soluplus, copolymers of acrylic and methacrylic acid, such as Eudragit L100-55, Eudragit L100, Eudragit EPO etc. The screening was done at various drug loading ranging from 5%-40%.

TABLE 2
Solubility Parameters calculations for HCV-4 API with various polymers

| Drug/Polymer | δ (van Krevelen) | δ (Hansen) | δ (Hoy) |
|---|---|---|---|
| HCV-4 | 27.0776 | 24.0865 | ~ |
| HPMC-AS | 26.0281 | 40.5648 | ~ |
| Kollidon PVP VA64 | 24.2935 | 25.6482 | 24.3355 |
| PVP (K30, K90 . . . ) | 25.9657 | 27.6816 | 24.8893 |
| Eudragit L100 | 23.447 | 21.1627 | 20.2345 |
| Eudragit L100-55 | 23.5976 | 20.4951 | 20.5515 |
| Eudragit EPO | 20.3652 | 16.68035 | 16.802 |
| Soluplus | 19.4651 | 20.2765 | — |

Theoretical calculations didn't predict any benefit of using one particular polymer over another in terms of providing stable amorphous dispersion.

### EXAMPLE 5-9

Amorphous Dispersion using HME Technology

| | Example | | | | | | | | | |
|---|---|---|---|---|---|---|---|---|---|---|
| | 5 | | 6 | | 7 | | 8 | | 9 | |
| Ingredients | Amounts | | Amounts | | Amounts | | Amounts | | Amounts | |
| Cpd A API | 4 | g | 4 | g | 4 | g | 4 | g | 4 | g |
| Eudragit | 16 | g | 0 | | 0 | | 0 | | 0 | |
| L100-55 | | | | | | | | | | |
| HPMC-AS | 0 | | 16 | g | 0 | | 0 | | 0 | |
| PVP-VA64 | 0 | | 0 | | 16 | g | 0 | | 0 | |
| Soluplus | 0 | | 0 | | 0 | | 16 | g | 0 | |
| Eurdagit | 0 | | 0 | | 0 | | 0 | | 16 | g |
| EPO | | | | | | | | | | |

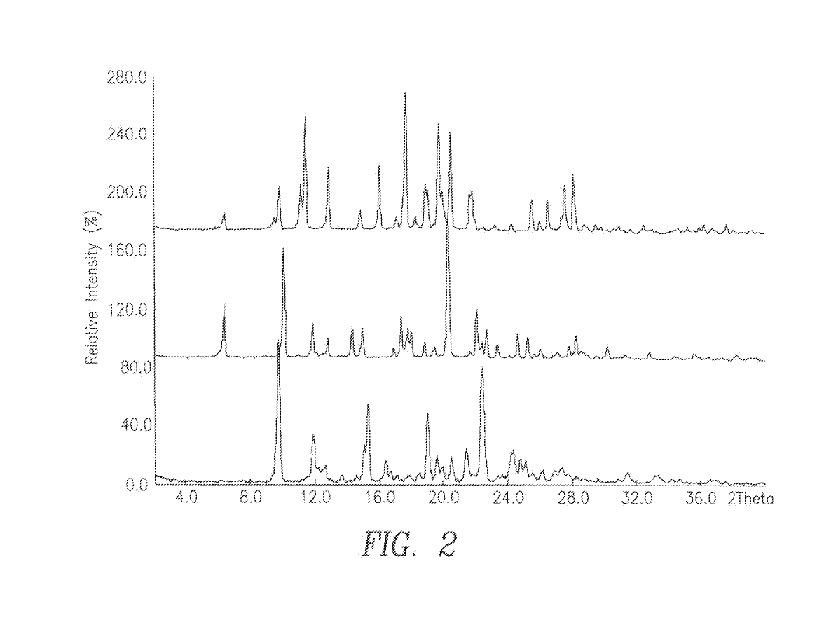

For the formulations 5-9, homogeneous blends were prepared using the turbular mixer. The formulations of Example 5-8 were processed using Leistriz Micro 18 lab scale extruder at constant feed rate of 10-15 g/min., screw speed of 150 rpm and processing temperature in the range of 160-190° C. The formulation of Example 9 was processed using processing temperature of 80-100° C. None of the formulation provided clear extrudes as drug didn't melt at the processing temperature range. All the formulations exhibited crystalline XRD pattern (see FIG. 2).

### EXAMPLE 10-13

Amorphous Dispersion Using HME Technology with Plasticizers

| | Example | | | | | | | |
|---|---|---|---|---|---|---|---|---|
| | 10 | | 11 | | 12 | | 13 | |
| Ingredients | Amounts | | Amounts | | Amounts | | Amounts | |
| Cpd A API | 2 | g | 2 | g | 2 | g | 1 | g |
| PVP-VA64 | 7.5 | g | 7.5 | g | 7.5 | g | 8.5 | g |
| Glyseryl Monosterate | 0.5 | g | 0 | | 0 | | 0 | |
| Stearic Acid | 0 | | 0.5 | g | 0 | | 0 | |
| Di-octyl sodium sulpho- | 0 | | 0 | | 0.5 | | 0 | |
| Succinate. | | | | | | | | |

Evaluation with plasticizers or lowering the drug loading up to 10% didn't provide any benefit for increasing the drug: polymer miscibility or solubility. All the formulations exhibited crystalline peak pattern by XRD.

### EXAMPLE 14-16

Amorphous Dispersion Using MBP Technology

Example

| | 14 | | 15 | | 16 | |
|---|---|---|---|---|---|---|
| Ingredients | Amounts | | Amounts | | Amounts | |
| Cpd A API | 1 | g | 1 | g | 1 | g |
| HPMC-P | 2.33 | g | 0 | | 0 | |
| Cellulose acetate | 0 | | 2.33 | g | 0 | |
| phthalate | | | | | | |
| Eudragit L100 | 0 | | 0 | | 2.33 | g |
| Dimethyl Acetamide | 15 | mL | 15 | mL | 15 | mL |
| (DMA) | | | | | | |

The drug and polymer were dissolved in the DMA by stirring at room temperature. The solution was then added to the temperature controlled anti solvent aqueous media (dilute HCl, pH 3.0) that allows rapid co-precipitation of drug and API. The residual DMA was extracted with frequent washing, followed by filtration and drying. All of the above formulation showed crystalline XRD pattern.

### EXAMPLE 17-20

Amorphous Dispersion Using MBP Technology

| | Example | | | | | | | |
|---|---|---|---|---|---|---|---|---|
| | 17 | | 18 | | 19 | | 20 | |
| Ingredients | Amounts | | Amounts | | Amounts | | Amounts | |
| Cpd A API | 1 | g | 0.5 | g | 1 | g | 1 | g |
| HPMC-AS LF | 2.33 | g | 0 | | 0 | | 0 | |
| HPMC-AS LF | 0 | | 9.5 | g | 0 | | 0 | |
| HPMC-AS HF | 0 | | 0 | | 4 | g | 0 | |
| Eudragit L100-55 | 0 | | 0 | | 0 | | 2.33 | g |

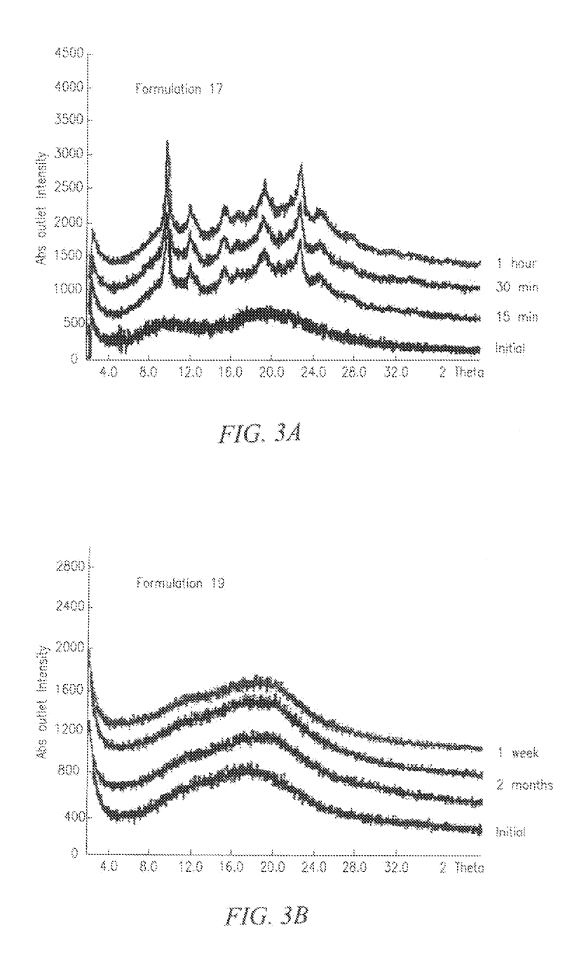

All the formulation with HPMC-AS based polymers and Eudragit L100-55 showed glass transition temperature in the range of 120-150° C. and amorphous PXRD pattern. The milled MBP powder of above formulations were subjected to accelerated stability studies by dispersing in aqueous based vehicle (pH 3.5±0.5) at room temperature and by exposing at 40° C. and 100% relative humidity (RH). The samples were analyzed for the presence of crystallinity, at different time points. All the HPMC-AS based formulations 17-19, even at the 5% drug loading level (i.e. formulation 18) started crystallizing out after 1 hour time point in aqueous vehicle, whereas the Eudragit L100-55 based formulation stayed amorphous for more than 60 days in the aqueous media. Similar trend was seen at extreme stress conditions of 40° C./100% RH (see FIGS. 3A and B).

These novel characteristics of the stabilized amorphous dispersion formulation of Compound A with Eudragit L100-55 are fully retained, when this composition was processed to drug formulations, e.g., tablets. In particular, direct compressing did not entail any change in the morphology.

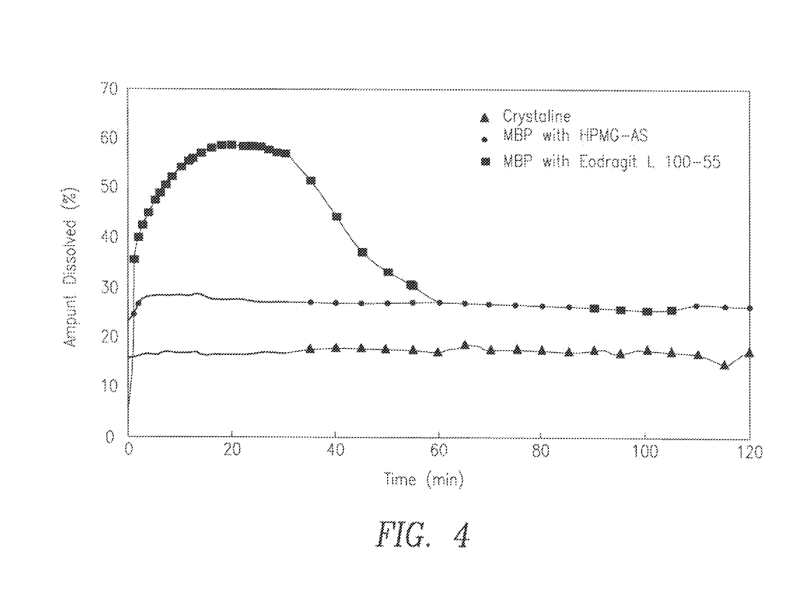

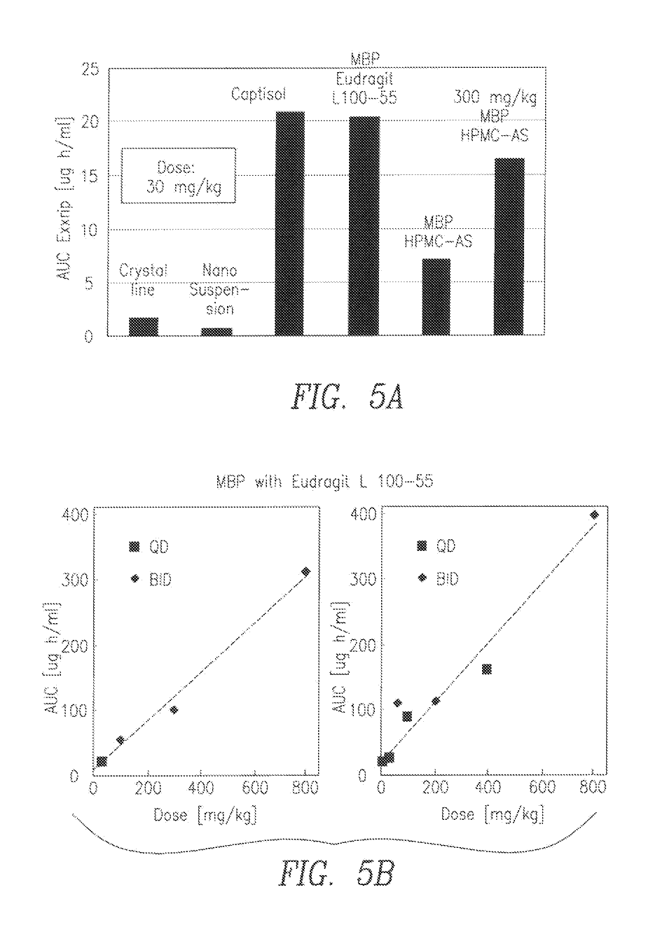

The structural characteristics of the amorphous compositions lead to greatly improved solubility kinetics and thus provided a significant increase in the bioavailability over crystalline form (see FIGS. 4 and 5A and B).

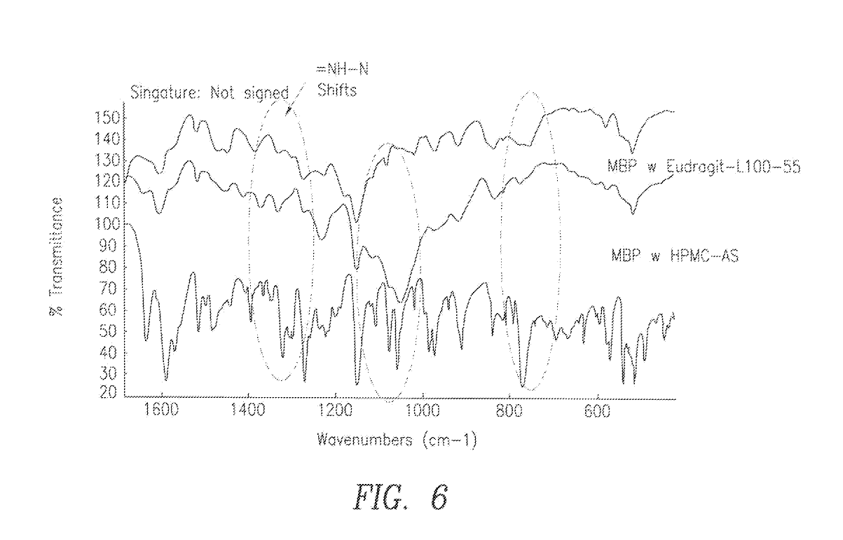

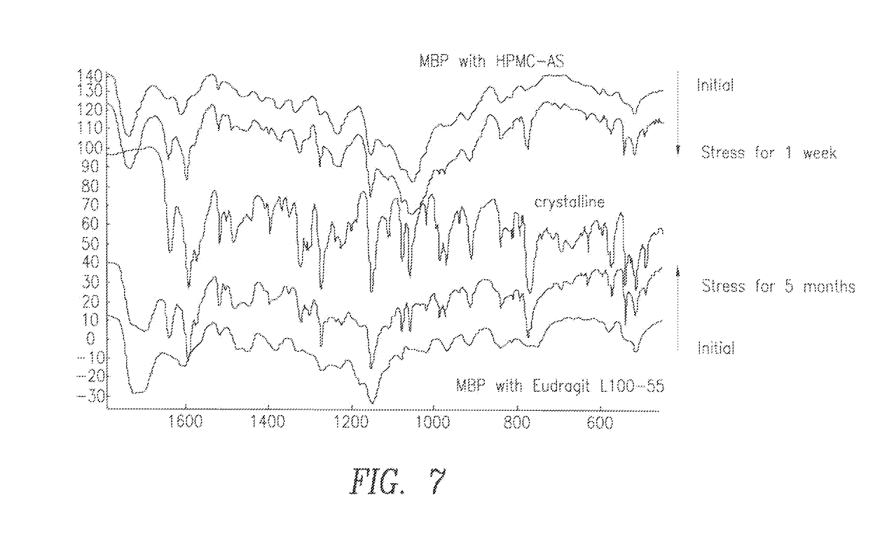

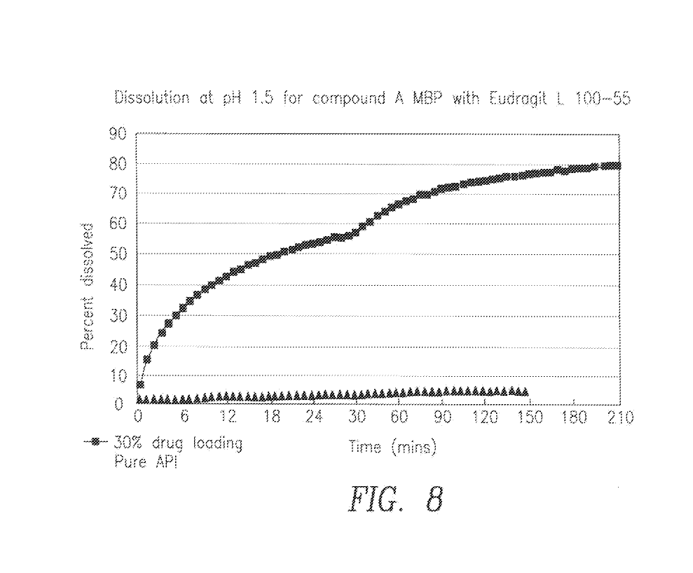

This extreme stability of amorphous dispersion with Eudragit L100-55 polymer was attributed to the ability of the drug to form specific interactions with the polymer via solvent mediated reaction i.e. in DMA, during MBP process. It has been found that the Compound A API has tendency to exhibit keto-enol tautomerism. This suggested that transition from a predominantly-keto to a predominantly-enol form occurs when HCV-4 API is dissolved in DMA, which could be the driving factor for availability of specific sites for interaction between drug and polymer. Those specific drug-polymer interaction were envisioned by infra-red spectra, collected by using a Golden Gate attenuated total reflection (ATR) accessory of Nexus 670 FT-IR spectrometer equipped with a XT-KBr beam splitter and a DTGS-KBr detector. The spectrum of the crystalline API was compared with amorphous dispersion obtained with HPMC-AS vs. Eudragit L100-55 (see FIG. 6) to elucidate the nature of the interactions. A number of differences were evident. In addition to a broadening of peaks, the amorphous sample with Eudragit L00-55 reflected significant changes in peak shape, intensity, and position. On the other hand HPMC-AS amorphous dispersion didn't exhibited significant shifts over the crystalline API. The recording of FTIR spectra as a function of stress at various time points facilitated to understand the extreme stability of Eudragit L100-55 amorphous dispersion (see FIG. 7). The FT-IR peak pattern marked the onset of crystallization event for amorphous HPMC-AS dispersion with-in 1-3 days. But for Eudragit L100-55, peak pattern shifted approximately after 5 months.

Another indication for existence of specific drug-polymer interactions was perceived from the unique dissolution behavior of Eudragit L100-55 amorphous dispersion under acidic conditions. Despite enteric nature of the Eudragit L100-55 polymer, significant amount of drug was releasing out in the dissolution media of pH 1.5 (˜80-90%), further substantiating the presence of in-situ ionic interaction between drug and polymer (see FIG. 8).

The above investigations reveal that the discovery of stable amorphous dispersion of Compound A with Eudragit L100-55 via MBP process was completely unforeseen and could not have been predicted based upon the previous experience with other compounds. Those specific drug-polymer interactions between Compound A API and Eudragit L100-55 formed only in the presence of solvent i.e. DMA, contrary to any of the previous experience and difficult to predict a priori. The existence of those specific interactions discouraged the rearrangement of drug molecules, thus retaining their disordered amorphous structure in stable amorphous dispersion.

### Purified Eudragit L100-55

In spite of higher bioavailability, superior solid state stability, the high dose predictions presented insurmountable difficulties in developing the drug product for the pre-clinical and clinical studies, because of the reported lower safety margins of Eudragit L100-55.

Eudragit L100-55 is commonly used functional excipients for the manufacturing of various pharmaceutical dosage forms. The anionic copolymer of methacrylic acid and ethyl acrylate possesses relative molecular mass of about 250,000 and contains 0.7% of sodium lauryl sulfate (SLS) and 2.3% of polysorbate 80, on weight basis of the dried powder composition.

The reported NOAEL for Eudragit L100-55 in dogs (Reference—safety study conducted by the supplier of Eudragit L100-55) is 100 mg/kg based upon 13-weeks safety study and 80 mg/kg based upon 52 weeks study. At higher doses, dose-dependent incidences of diarrehea and weight losses were observed for all the animals. Severe instances of emesis and the presence of blood in the feces were also evident in animals. No cause/s for these GI toxicities has been identified by the vendor or in the literature. Similar GI effects in dogs were observed during pre-clinical safety study of Compound A, for the highest dose group and corresponding placebo dose group at a dose of 1.8 g of Eudragit L100-55/kg/day for 3 weeks.

Since the safety of Eudragit L100-55 limits the daily dose of Compound A which can be evaluated either in the pre-clinical studies or eventually in the clinical studies, efforts were made to understand the cause of observed GI toxicities. For the first time unexpectedly, it was found that these untoward toxicities of Eudragit L100-55 are associated with the presence of SLS in the original polymer.

For example, a process was developed to purify the Eudragit L100-55 using rinsing/washing method. The polymer was slurried in water at 5-15% solid content level at room temperature (to 25° C.-32° C.). The surfactant being soluble in water, extracts out in water over the period of time. The resultant solid mass was separated either via filtration followed by drying or spray drying of the dispersion. The drying conditions were suitably chosen to provide the desired moisture levels of less than 5%. The particle size of the dried material was tailored to be a particular size as dictated by the end usage. The SLS level was lowered, up to 0.05%-0.1% level in purified Eudragit L100-55.

When this purified Eudragit L100-55 was tested in in two-week tolerability study in dogs, none of the earlier reported overt GI signals i.e. diarrhea, blood in the feces, decreased food consumption and body weight etc. were seen, even at the 1.8 g/kg/day dose level. The removal of the surfactant, especially SLS facilitated the establishment of higher safety margins for Eudragit L100-55, enabling its use at much higher doses than previously established limits.

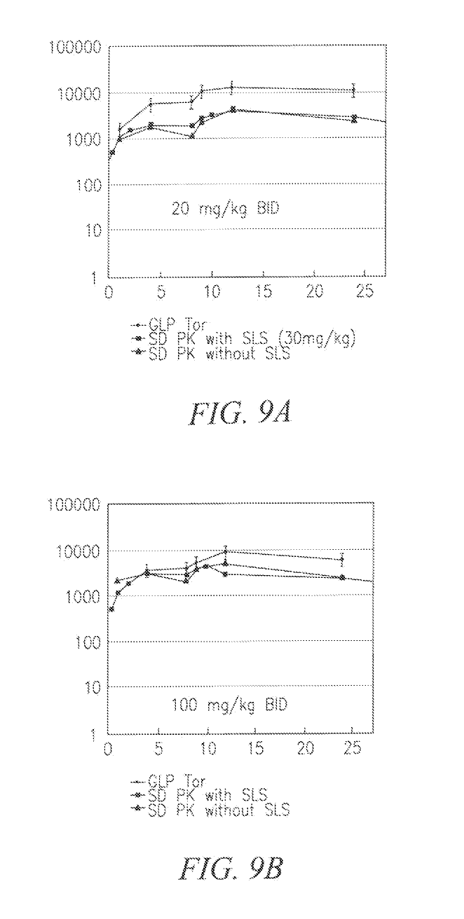

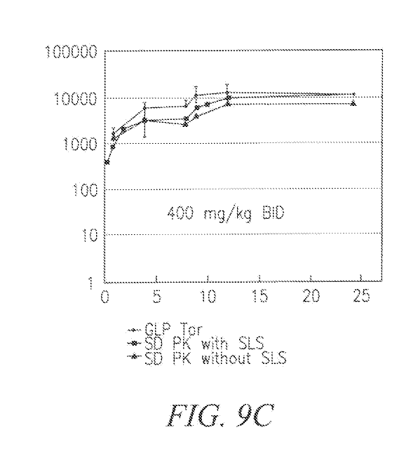

Furthermore, a stable amorphous formulation of the Compound A has been developed using purified Eudragit L100-55, to support the high dose requirement for pre-clinical safety studies (and human studies) of Compound A. The removal of surfactant didn't cause any change on the in-vitro and in-vivo performance of amorphous formulation with Eudragit L100-55. The PK/exposure profiles in animals were comparable (see FIGS. 9A, B and C).

Purified Eudragit L100-55 provided significant advantage in terms of extending the safety limits for Eudragit L100-55 without impacting the polymer performance and thus enabling the usage of the polymer at much higher level for other compounds as well.

What is claimed:

1. A pharmaceutical formulation comprising a physically stable, amorphous solid dispersion comprising a compound having an aqueous solubility of less than 1 μg/ml. with a melting point of >270° C., together with an ionic polymer.

2. A pharmaceutical formulation in accordance with claim 1, wherein said ionic polymer is an anionic copolymer of methacrylic acid and ethyl acetate.

3. A pharmaceutical formulation in accordance with claim 1, wherein said compound having an aqueous solubility of less than 1 μg/ml. is N-{4-[6-tert-butyl-5-methoxy-8-(6-methoxy-2-oxo-1,2-dihydro-pyridin-3-yl))-quinolin-3-yl]-phenyl}-methanesulfonamide.

4. A composition comprising an anionic copolymer of methacrylic acid and ethyl acetate having a relative mass of about 250,000 and containing 0.7% of sodium lauryl sulfate (SLS) and 2.3% of polysorbate 80, on a weight basis of the dried powder composition and N-{4-[6-tert-butyl-5-methoxy-8-(6-methoxy-2-oxo-1,2-dihydro-pyridin-3-yl))-quinolin-3-yl]-phenyl}-methanesulfonamide.

5. A composition in accordance with claim 4, wherein said anionic copolymer contains less than 0.1%, on a weight basis of the dried powder composition, of sodium lauryl sulfate (SLS).

6. A composition in accordance with claim 5, wherein said anionic copolymer contains from about 0.05% to 0.1%, on a weight basis of the dried powder composition, of sodium lauryl sulfate (SLS).

7. The use of the composition according to claim 4 for the treatment or prophylaxis of hepatitis C.

8. The use of the composition according to claim 5 for the treatment or prophylaxis of hepatitis C.

9. The use of the composition according to claim 6 for the treatment or prophylaxis of hepatitis C.

10. A method for preparing a physically stable amorphous solid dispersion of a compound having an aqueous solubility of less than 1 μg/ml and an ionic polymer which comprises forming a solution of the compound and the polymer in dimethyl acetamide and co-precipitating the compound with the polymer using anti-solvent.

11. A method in accordance with claim 10, wherein said compound having an aqueous solubility of less than 1 μg/ml. is N-{4-[6-tert-butyl-5-methoxy-8-(6-methoxy-2-oxo-1,2-dihydro-pyridin-3-yl))-quinolin-3-yl]-phenyl}-methanesulfonamide and said ionic polymer is an anionic copolymer of methacrylic acid and ethyl acetate having a relative mass of about 250,000 and containing 0.7% of sodium lauryl sulfate (SLS) and 2.3% of polysorbate 80, on a weight basis of the dried powder composition.

12. A method in accordance with claim 11 additionally including the step of treating said copolymer of methacrylic acid and ethyl acetate to reduce the content of sodium lauryl sulfate (SLS) to less than 0.1%, on a weight basis of the dried powder composition, prior to forming said solution.
